SEQUENCE LISTING (16 sequences)

SEQ ID NO: 1 - DNA, 264 bp, Peronospora tabacina
atctacaccttttaggcaaagcttcagttttatcttcatgaaaataaataaatagataagacctaaaattaattggtgaaatgatgtggaaaattattattttttctttttcttatcctttggcatcagttttaagaaccttcatctatcttcactactataaattgaggtcctttcctcagccattacatcaacaattcattatagcatttgtatagaaacaaacttgtgtacactaaacaaaaatattgagttctagccatg

SEQ ID NO: 2 - DNA, 823 bp, Peronospora tabacina
tctcattggggaccttgctctcatcggagaatggctacaccttctctcagtagcgattttctccattaatcttggttctaacctgagttgctttgtctattcttacctctgttcgaaaatcgaaataaaaactaggcttttgctcttgaatcggtatagaagattagggttttgacaagagagagaattttatatgtattcagatgaagaaatggagaagaaaggtgaaaaaataaaaaagaaaggtaaaaggaagaggaaggaaagtgaaatataaagaaaaaaagagaaaataaaaaaaaaatttaaaaatgttaggctaattagccattaaacactgtcacgtgagccatttcaatggcttttttcaaccgtcatgcgctctggcaatttattacactcattatgccacttaagtgacgaaagcgttttatttgaaggggatatagttcaagagtttttggacatgcgatagtttaggtaggcaactcataaaagttgtatagtttaagcgagttttagggcactaactcttttgaaattgatctaaaaatgactacatattgttttggttattatttgcatcaaataacttacagtttttaaaaaatactcaaaggattgtttttcatgggcaccaggaaatgaaagccatgtgtatttacattatataaatgaaacaaagtcgtcttcgatggctcgatgtcgagtcattctcatgtgttgttttattagatggctcatctgctataaatagatgcataagataacactctccatcctcaaaaagaaaaacagtaaactcaggagatagtaagttatg

SEQ ID NO: 3 - DNA, 436 bp, Peronospora tabacina
ctggagtaataaaataaggtgaggcgtataaaacttaatcatgattaagcgaaaacaatttatgtgcaaatgcgtatccattattttgggtaccactcaaatgattttgtctttcctaaaacccgagcatatccttttttttgtttccatcatttgtccaatactcgcatgggagagcgactaaattcgtatttgaaaatttcacattaagaggtaagtgctccctaataaaagtaaatttaatttcatacccaagaccagagatttttaaaagaagcatacctactcttggcatcaactatataagtagtctagcatgagacgtggagatcagtatactagcctttaattgaggatcaccatatacgtacccaataaaactcctaaactctcttccatttctttgatcaacattcattcttgatttccgacgatg

SEQ ID NO: 4 - DNA, 1378 bp, Peronospora tabacina
ccttttatacggggtggatggttgtgtcttggtcgttggattgccattgatcaacggctgtgatctttccattaaaagaaacggtgtcgtttgctttcatactggggtggatcagaccgggtttcattgggtcgggtctgatagcgggtaaaggagatgggacgagggctgttagatcagcctggtttgaacggctgagattagacggccttatacggcgtcgtttgggtaaaggattggcctgatctgggccgttcctttgaaccaatcaacggctcgaagagatgatgcctatacggcgtcgtttggggcgtcttgcagaaggcctggttggactgggtcatttgcattggtttgggcccgattttatttaaaatcttggcccaaatctaatgttttccttcttataattaaataaaaattcctaataattaaaaaaaaaaatcataccaatattaataaatactcaaaacaacaattatcactcacattgacatttaattaaaaataaaatcattaaatgacaacaaaaagtagaataaaagacacatatattttacgattttccttttaaaatcaattacggttcaaatacacacgacacacattttttttatattttgtttgataaagaataaaaatagacgaaatcacaaataactaacaaaatgccacgtaaaaaatccaaaaattgtacagcaagaccatttgttattatttttatttcttttagagtgattgtcgcgtaaaacaaaaatcacgtgctcacaatcctaatgatatatgaaaaaataaaaactagaagctaaacatcatattaataatagcgttggacttatgtggagataaaatctctttgacacagccaaaacaatccgttaatgtcgtcatgttcaaaatcaacatattttaaattatttttaattctctacatatgtcgaatttgagacaacaaatgcatcctcttggttaggtaaatacttacattcatcgaatatttatatcaacctaatatatacatcacataagtctttataagcatggctatcaattaatggacaacacttccacatttctttcgtcttcgaatttaaaatggacagtgttttaaggcatatatccacgcacaaggcaaactcatacttatgctcctgtacaccatatgcagacagcacctcaacattgaccggttttgaattgactacattcgcgccaacttgtgcattataatcttgtcaatgcaaaaatttaaaagatacggatgaacagtactattcataataactaattaattatccatcgccatccacctataaatacggatagcaagaaactaaggatcatatcatcaaacagctaaaagaagaaattaaacatg

SEQ ID NO: 5 - DNA, 958 bp, Peronospora tabacina
attcgagtatgttggtgtacatacaagtatttttttttgggttttcaatcggcgtccagtatcagtattgaagyccgattaaattcgaattcgcaccgaaaaatactacattaggagataaaacgccccctgataatagttagtaatgcaaaattataatatatatatatatatgtgtgtgtgtgtgtgtgtgtgtgtgtgttttactttttcagcattaaatttcatataaaaaatgcaagaattttctcataaattttcctaatgatatatgaaaaaataaaaactagaagctaaacatcatattaataatgtgttggacttatgtggagataaaatctctttgacacagccaaaacaatccgttattgtcgtcatgttcaaaatcaacatattttaaattatttttaattcgctacatatgtcaaatttgagacaacaaatgcatactcttggttaggtaaatacttacattcatcgaatatttatatcaacctaaatcacatatgtctttataagcatggctatcaattaatagtagttaatttaatacttattcttatcttatttatcacttacctgtgatgaattaatataggttggtataaaaacccgaactcctcggtgactcagtctctgacaacacttccacatttctttcgtcttcgaatttaaaatggacagtgttttaaggcatatatccacgcacaaggtaagctcatacttatgctcctgtacaccatatgcagacagcrcctcaacattgacggttttgaattgactacatttgcgccaacttgtgcattataatcttgtcaatgcaaaaatttaaaagatacggatgaacagtactattcataataactaattaattatccatcgccatccatctataaatacggatagcaagaaactaaggatcatatcatcaaacagctaaaagaagaaattaaacatg

SEQ ID NO: 6 - DNA, 27 bp, Artificial Sequence
  Chemically synthesized
gctcctcagtcactcctttgcccatag

SEQ ID NO: 7 - DNA, 27 bp, Artificial Sequence
  Chemically synthesized
gactgaggcaaggacctcaatttatag

SEQ ID NO: 8 - DNA, 32 bp, Artificial Sequence
  Chemically synthesized
gccttggcatctgcatgttccacattgcttac

SEQ ID NO: 9 - DNA, 34 bp, Artificial Sequence
  Chemically synthesized
ggacgagagcaaggaaactaactctgtgaacagc

SEQ ID NO: 10 - DNA, 29 bp, Artificial Sequence
  Chemically synthesized
gctctagacattggggaccttgctatcgg

SEQ ID NO: 11 - DNA, 33 bp, Artificial Sequence
  Chemically synthesized
gcggatcccttactatctcctgagtttactgtt

SEQ ID NO: 12 - DNA, 31 bp, Artificial Sequence
  Chemically synthesized
gctctagagggagctctcccgcctggtctac

SEQ ID NO: 13 - DNA, 32 bp, Artificial Sequence
  Chemically synthesized
gcaagcaagcaacagagccaagcaactgagca

SEQ ID NO: 14 - DNA, 32 bp, Artificial Sequence
  Chemically synthesized
caccaacaaacgcaccaagtgacagcagggtt

SEQ ID NO: 15 - DNA, 31 bp, Artificial Sequence
  Chemically synthesized
cccggctcacaagcttattcgggcctgacaa

SEQ ID NO: 16 - DNA, 35 bp, Artificial Sequence
  Chemically synthesized
ggcctgacaagcggagagattggcccactaaaaga

(19) United States
(12) Patent Application Publication
(10) Pub. No.: US 2004/0205841 A1
(43) Pub. Date: Oct. 14, 2004

(54) Pathogen induced promoters

(76) Inventor: Christopher Lawrence, Blacksburg, VA (US)

Correspondence Address: MCDERMOTT, WILL & EMERY, 600 13th Street, N.W., Washington, DC, 20005-3096, US

(21) Appl. No.: 10/638,464

(22) Filed: Aug. 12, 2003

Related U.S. Application Data
(60) Provisional application No. 60/402,528, filed on Aug. 12, 2002.

(51) Int. Cl. C12N 15/82; C07H 21/04; A01H 1/00; C12N 15/87; C12N 15/63

(52) U.S. Cl. 800/279; 536/24.1; 435/320.1; 435/468; 800/278

(57) ABSTRACT

Pathogen-inducible plant promoters are identified. The promoters control expression of “hypersensitive response” proteins, including a protease inhibitor, an isocitrate lyase, and a glycoprotein. Heterologous gene sequences are produced by operably linking a promoter according to the present invention with a gene to be expressed in a transformed plant. Transformed plants are made by transforming a vector with a heterologous gene according to the present invention and then transforming the plant with the transformed vector. The transformed plants are capable of expressing a pre-selected protein in response to challenge by a plant pathogen, for example tobacco blue mold—P. tabacina.

### RELATED APPLICATIONS

This application relates to and claims priority to U.S. Serial No. 60/402,528, filed Aug. 12, 2002, incorporated herein in its entirety.

### FIELD OF THE INVENTION

The present invention relates to isolated DNA sequences comprising promoter regulatory regions of plant genes, which are inducible during pathogen infection of plants. Specifically, the invention relates to promoters of tobacco genes that are inducible by pathogens, such as tobacco blue mold. In particular, the promoters are inducible by infection with Peronospora tabacina.

### BACKGROUND OF THE INVENTION

Tobacco blue mold, or Peronospora tabacina Adam, is an infectious agent that causes destruction of tobacco plants throughout the Americas, and gives rise to local and regional epidemics. Commercial tobacco is a seasonal crop that is grown in the temperate, warm and cool, humid farming zones of the Southeastern and Eastern United States, Canada, and Caribbean Basin countries. Following the winter of each year, tobacco in the United States is exposed to asexual, windborne sporangiospores originating from commercial tobacco and wild Nicotiana species in the tropical zones south of the 30th parallel. As the fungus does not overwinter in more temperate zones, fresh outbreaks of tobacco blue mold are caused by newly introduced inoculum during each growing season. Blue mold disease poses a particularly ominous threat to tobacco growing areas of Kentucky, costing growers tens of millions of dollars in annual losses of marketable leaf yield. As transgenic tobacco has become an increasingly important source of biopharmaceuticals, tobacco blue mold also poses a major threat to molecular agricultural operations. It is thus very desirable to control tobacco blue mold.

Tobacco blue mold reproduces by means of both sporangiospores and oospores. The sporangiospores are released from sporangiophores on the underside of tobacco leaves, and are borne by the wind. Depending upon atmospheric conditions, sporangiospores may be carried long distances by wind. Tobacco blue mold may also be spread on transplanted seedlings. Windborne epidemics of tobacco blue mold tend to advance along well-defined epidemic fronts. However, long distance, e.g. over 100 km, transport of blue mold infection has been documented.

Forecasting methods have been developed to assist tobacco farmers in predicting the spread of tobacco blue mold epidemics. Equipped with such knowledge, a tobacco farmer may employ various prophylactic measures to protect a tobacco crop. Such measures may include application of fungicides to the tobacco crop. However, residual fungicides both on the tobacco leaves and in the environment are undesirable, and there is increasing market, societal and governmental regulatory pressure to limit or eliminate the use of conventional chemical fungicides on crop plants. Additionally, high costs are associated with prevention of blue mold disease using fungicides. It is therefore desirable to develop a method of combating tobacco blue mold that does not require the use of chemical fungicides.

Plants have inherent mechanisms for preventing and combating pathogens. Plants defend themselves against pathogenic infections by generating a “hypersensitive response”, which results in the synthesis of proteins that prevent the spread of pathogens from infected cells to neighboring, healthy cells. As part of the response, pathogen-free cells also become resistant to other pathogens that were not the causative agents of the primary infection. The genes that are activated during such a response include those encoding for hydroxyproline-rich glycoproteins (HRGP), pathogenisis related proteins (PR), and various enzymes that catalyze the synthesis of aromatic compounds. However, in a susceptible host, no induction of defense response is seen until significant damage is done to the host tissue. Given the virulence of tobacco blue mold, it would be desirable to evoke the plant's defense response at the earliest stages of infection, thereby avoiding the spread of irreparable damage caused by the pathogen.

Plants also have mechanisms for responding to damage caused by environmental factors other than microbes. Plants respond to injury by activating a number of genes whose protein products enable the plant to defend itself against further insult. Typically, the defensive response is rapid, and is mediated by the activation of promoters by biological, physical, and/or chemical damage. For example, heat shock proteins are expressed in response to an increase in temperature; protease inhibitors are expressed in response to injury by insects, and pathogen-related proteins are expressed in response to aromatic compounds, such as salicylic acid. The heat shock proteins enable the plant to withstand increased temperatures; the protease inhibitors enable the plant to specifically inhibit the digestive enzymes of the insects and render the plant resistant to insect damage; and pathogen-related proteins may render the plant more resistant to viral infection.

Studies have described inducible promoters of plant genes that are activated during the hypersensitive response. For example, U.S. Pat. No. 5,689,056 describes the HMG2 promoter elements of the HMGR (3-hydroxy-3-methylgluttaryl CoA reductase) genes that are activated by pathogens, pests, wounding, elicitor and chemical treatments. U.S. Pat. No. 5,057,422 describes the promoter of a glycine rich protein (GRP) gene of Nicotina tabacum, which is inducible by salicylate, as well as other aromatic compounds including acrylic acid, ethylene, ethephone. U.S. Pat. No. 5,608,143 describes the cloning and isolation of promoter fragments derived from corn, petunia and tobacco, which are highly responsive to substituted benzene sulfonamides and related compounds. The three patents also describe how the inducible plant promoters can be used for genetically engineering plants that are resistant to a variety of diseases and pests.

It would be desirable to produce plants that are stably transformed with a P. tabacina-inducible promoter of tobacco plants to control genes that confer resistance to multiple diseases. In addition, it would be desirable to use the P. tabacina-inducible tobacco plant promoters to drive the expression of heterologous genes for producing proteins or secondary metabolites that may be useful therapeutic agents.

### SUMMARY OF THE INVENTION

The foregoing and other needs are met by embodiments of the present invention, which provides isolated DNA sequences encoding pathogen-inducible plant promoters.

The foregoing and other needs are further met by embodiments of the present invention, which provide a method of preparing a transgenic plant, said transgenic plant having been transformed by a promoter DNA sequence inducible by a plant pathogen, such as P. tabacina. The inventive method comprises: providing a DNA consisting of a plant pathogen-inducible promoter operably linked to a gene or poltnucleotide sequence of interest; and transforming a plant with the recombinant construct; the foregoing steps resulting in production of said transgenic plant.

The foregoing and other needs are met by embodiments of the present invention, which provide a transformed plant, said plant having been transformed to express an exogenous gene comprising a P. tabacina-inducible promoter.

An objective of the present invention is to identify pathogen-inducible gene promoters, in particular P. tabacina-inducible promoters.

Another objective of the invention is to use the identified cDNAs to isolate the promoter sequences of other pathogen-inducible plant promoters.

Another objective is to generate promoter-reporter gene constructs to identify and isolate optimal elements of the inducible promoters. The optimal promoter sequences may be fused to DNAs that encode plant defense genes, as well as other genes of interest to create genetic constructs that can be used to confer resistance to pathogens in tobacco. In some embodiments of the present invention, optimal promoter fragments are identified in tobacco plants infected with P. tabacina.

Another objective of the invention is to operably link the optimal fragments of the inducible promoters to heterologous genes that encode plant defense response elicitors as well as other genes of interest.

Another objective of the invention is to use the promoter-heterologous gene constructs according to the present invention to generate transgenic plants that are resistant to a variety of plant diseases.

Another objective of the invention is to use promoter-heterologous gene constructs according to the present invention to generate transgenic that are capable of producing biologically or pharmacologically relevant proteins.

Other objects and advantages of the present invention will become apparent upon consideration of the description, drawings and claims provided herein.

### DETAILED DESCRIPTION OF THE INVENTION

The present inventor has discovered five plant pathogen-inducible tobacco promoter sequences of the genes encoding a protease inhibitor (PI), isocitrate lyase (ICL), membrane (clone 80) intrinsic protein, or a novel glycoprotein. The promoters are each induced within 24 hours following infection of plants with a plant pathogen, such as the downey mildew disease-causing pathogen, Peronospora tabacina. The invention provides isolated polynucleotides encoding the plant pathogen-inducible promoters, and a method for using the promoters for conferring pathogen or disease resistance in plants, and in a preferred embodiment, resistance to P. tabacina in tobacco. Modifications of the plant pathogen-inducible promoters are also included in the invention.

Some embodiments of the present invention comprise the process for identifying plant genes having promoters that are inducible by infection with plant pathogens, particularly P. tabacina or other downey mildew disease causing pathogens. By “promoter” is intended a regulatory region of DNA usually comprising a TATA box capable of directing RNA polymerase II to initiate RNA synthesis at the appropriate transcription initiation site for a particular coding sequence. A promoter can additionally comprise other recognition sequences generally positioned upstream or 5′ to the TATA box, referred to as upstream promoter elements, which influence the transcription initiation rate. It is recognized that having identified the nucleotide sequences for the promoter regions disclosed herein, it is within the state of the art to isolate and identify further regulatory elements in the 5′ untranslated region upstream from the particular promoter region identified herein.

The promoter sequence may include enhancer sequences, which enhance promoter activity. It is recognized that to increase transcription levels, enhancers can be utilized in combination with the promoter regions of the invention. Enhancers are known in the art and include the SV40 enhancer region, the 35S enhancer element, and the like.

The nucleotide sequence for the promoters of the invention, as well as functional fragments and variants thereof, can be provided in expression cassettes along with heterologous nucleotide sequences for expression in the plant of interest. Such an expression cassette may be provided with a plurality of restriction sites for insertion of the nucleotide sequence to be under the transcriptional regulation of the pathogen-inducible promoter. These expression cassettes are useful in the genetic manipulation of any plant to achieve a desired phenotypic response.

The genes or nucleotide sequences of interest expressed by the pathogen-inducible promoter of the invention can be used for varying the phenotype of the plant. This can be achieved by increasing expression of endogenous or exogenous products in the plant upon infection by blue mold or other downey mildew, for example.

General categories of genes of interest for the purposes of the present invention include, for example, those genes involved in information, such as zinc fingers, those involved in communication, such as kinases, and those involved in housekeeping, such as heat shock proteins. More specific categories of transgenes include genes encoding important traits for agronomic quality, insect resistance, disease resistance, and herbicide resistance. Still other categories of transgenes include genes for inducing expression of exogenous products such as enzymes, cofactors, and hormones from plants and other eukaryotes, as well as prokaryotic organisms. It is recognized that any gene of interest, including the native coding sequence, can be operably linked to the promoter of the invention and expressed in the plant upon induction of the promoter.

The invention also comprises optimal DNA sequences of the P. tabacina-inducible promoters. An optimal promoter sequence is a promoter sequence whose activation by P. tabacina occurs rapidly and to a high level when compared to a baseline level. Baseline levels are those detected prior to induction by the pathogen. Optimal promoter regions of each of the P. tabacina-inducible promoters can be obtained for example by deleting, rearranging, mutagenizing the promoter or fragments thereof and/or by insertion of plant promoter enhancer elements. Optimal promoter DNA can be operably linked to a reporter gene, such as the GUS gene, and the resulting promoter-reporter construct transferred into a plant, such as tobacco or Arabidopsis plants, preferably using A. tumefaciens technology. The constructs may be used to transform any plant, in particular a plant susceptible to downey mildew disease.

Operably linked refers to the fusion of the DNA sequence of a promoter, promoter fragment, or optimal promoter fragment to a DNA encoding a protein in such an orientation that a functional RNA is transcribed and translated into a functional protein. The activity of each of the modified promoters can be assayed for the presence of the reporter protein in plants, as described above.

The isolated promoter sequences of the present invention can be modified to provide for a range of expression levels of the heterologous sequence. Less than the entire promoter region can be utilized and the ability to drive pathogen-inducible expression retained. However, it is recognized that expression levels of mRNA can be decreased with deletions of portions of the promoter sequence. Thus, the promoter can be modified to be a weak or strong promoter. Generally, by “weak promoter” is intended a promoter that drives expression of a coding sequence at a low level. By “low level” is intended levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts.

In another embodiment of the invention, an isolated pathogen-inducible promoter sequence of the invention having a nucleotide sequence as set forth in SEQ ID NO. 1, SEQ ID NO. 2, SEQ ID NO. 3, SEQ ID NO. 4 OR SEQ ID NO. 5 is used to isolate other plant pathogen-inducible promoters having a nucleotide sequence that has at least 90% sequence similarity thereto. In a preferred embodiment of the invention, an isolated pathogen-inducible promoter sequence of the invention having a nucleotide sequence as set forth in SEQ ID NO. 1, SEQ ID NO. 2, SEQ ID NO. 3, SEQ ID NO. 4 or SEQ ID NO. 5 is used to isolate a DNA molecule that hybridizes under stringent conditions with the DNA sequence of SEQ ID NO. 1, SEQ ID NO. 2, SEQ ID NO. 3, SEQ ID NO. 4 or SEQ ID NO. 5, wherein the stringent conditions comprise hybridization at about 65 degree C. followed by washing for about one hour in 2×SSC buffer at about 65 degree C., then washing for about 30 minutes in 0.2×SSC buffer at about 65 degree C.

Finally, optimal promoter fragments can be fused with genes encoding plant defense response elicitors, as well as any other gene of interest. These genes can be introduced into plants to generate transgenic plants having enhanced resistance to multiple diseases, for example. In addition, these promoters may be useful to control the expression of genes for applications other than for conferring disease resistance, such as the production of antibodies, biopharmaceuticals and edible vaccines, and other products of molecular farming.

Insect resistance genes that may be operably linked to the promoters of the invention may encode resistance to pests that have great yield drag such as rootworm, cutworm, European Corn Borer, and the like. Such genes include, for example, Bacillus thuringiensis endotoxin genes, U.S. Pat. Nos. 5,366,892; 5,747,450; 5,737,514; 5,723,756; 5,593,881; Geiser et al. (1986) Gene 48:109; lectins, Van Damme et al. (1994) Plant Mol. Biol. 24:825; and the like.

Genes encoding disease resistance traits include detoxification genes, such as against fumonosin (PCT/US95/10284 filed Jun. 7, 1995); avirulence (avr) and disease resistance (R) genes. Jones et al. (1994) Science 266:789; Martin et al. (1993) Science 262:1432; Mindrinos et al. (1994) Cell 78:1089; and the like.

Alterations in gene expression may also affect the type or amount of products of commercial interest; for example, starch for the production of paper, textiles and ethanol. Another important commercial use of transformed plants is the production of polymers and bioplastics such as described in U.S. Pat. No. 5,602,321 issued Feb. 11, 1997. Genes such as B-Ketothiolase, PHBase (polyhydroxybutyrate synthase) and acetoacetyl-CoA reductase (see Schubert et al. (1988) J. Bacteriol 170(12):5837-5847) facilitate expression of polyhyroxyalkanoates (PHAs).

An expression cassette of the invention of the invention preferably includes at the 3′ terminus of the heterologous nucleotide sequence of interest, a transcriptional and translational termination region functional in plants. The termination region can be native with the promoter nucleotide sequence of the present invention, can be native with the DNA sequence of interest, or can be derived from another source. Convenient termination regions are available from the Ti-plasmid of A. tumefaciens, such as the octopine synthase and nopaline synthase termination regions. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. 1989) Nucleic Acids Res. 17:7891-7903; Joshi et al. (1987) Nucleic Acid Res. 15:9627-9639.

The expression cassettes can additionally contain 5′ leader sequences. Such leader sequences can act to enhance translation. Translation leaders are known in the art and include: picomavirus leaders, for example, EMCV leader (Encephalomyocarditis 5′ noncoding region), Elroy-Stein et al. (1989) Proc. Nat. Acad. Sci. USA 86:6126-6130; potyvirus leaders, for example, TEV leader (Tobacco Etch Virus), Allison et al. (1986); MDMV leader (Maize Dwarf Mosaic Virus), Virology 154:9-20; human immunoglobulin heavy-chain binding protein (BiP), Macejak et al. (1991) Nature 353:90-94; untranslated leader from the coat protein mRNA of alfalfa mosaic virus (AMV RNA 4), Jobling et al. (1987) Nature 325:622-625); tobacco mosaic virus leader (TMV), Gallie et al. (1989) Molecular Biology of RNA, pages 237-256; and maize chlorotic mottle virus leader (MCMV) Lommel et al. (1991) Virology 81:382-385. See also Della-Cioppa et al. (1987) Plant Physiology 84:965-968. The cassette can also contain sequences that enhance translation and/or mRNA stability.

In those instances where it is desirable to have the expressed product of the heterologous nucleotide sequence directed to a particular organelle, particularly the plastid, amyloplast, or to the endoplasmic reticulum, or secreted at the cell's surface or extracellularly, the expression cassette can further comprise a coding sequence for a transit peptide. Such transit peptides are well known in the art and include, but are not limited to, the transit peptide for the acyl carrier protein, the small subunit of RUBISCO, plant EPSP synthase, and the like.

In preparing the expression cassette, the various DNA fragments can be manipulated, so as to provide the DNA sequences in the proper orientation and, as appropriate, in the proper reading frame. Toward this end, adapters or linkers can be employed to join the DNA fragments or other manipulations can be involved to provide for convenient restriction sites, removal of superfluous DNA, removal of restriction sites, or the like. For this purpose, in vitro mutagenesis, primer repair, restriction digests, annealing, and resubstitutions, such as transitions and transversions, can be involved.

The present invention also provides vectors capable of expressing genes of interest under the control of the pathogen-inducible promoter. In general, the vectors should be functional in plant cells. At times, it may be preferable to have vectors that are functional in E. coli (e.g., production of protein for raising antibodies, DNA sequence analysis, construction of inserts, obtaining quantities of nucleic acids). Vectors and procedures for cloning and expression in E. coli are discussed in Sambrook et al. (1974).

The transformation vector comprising the promoter sequence of the present invention operably linked to a heterologous nucleotide sequence in an expression cassette can also contain at least one additional nucleotide sequence for a gene to be cotransformed into the organism. Alternatively, the additional sequence(s) can be provided on another transformation vector.

Vectors that are functional in plants can be binary plasmids derived from Agrobacterium. Such vectors are capable of transforming plant cells. These vectors contain left and right border sequences that are required for integration into the host (plant) chromosome. At minimum, between these border sequences is the gene to be expressed under control of the pathogen-inducible promoter. In preferred embodiments, a selectable marker and reporter gene are also included. For ease of obtaining sufficient quantities of vector, a bacterial origin that allows replication in E. coli is preferred.

Reporter genes can be included in the transformation vectors. Examples of suitable reporter genes known in the art can be found in, for example, Jefferson et al. (1991) in Plant Molecular Biology Manual, ed. Gelvin et al. (Kluwer Academic Publishers), pp. 1-33; DeWet et al. (1987) Mol. Cell. Biol. 7:725-737; Goff et al. (1990) EMBO J. 9:2517-2522; Kain et al. (1995) BioTechniques 19:650-655; and Chiu et al. (1996) Current Biology 6:325-330.

Selectable marker genes for selection of transformed cells or tissues can be included in the transformation vectors. These can include genes that confer antibiotic resistance or resistance to herbicides. Examples of suitable selectable marker genes include, but are not limited to, genes encoding resistance to chloramphenicol, Herrera Estrella et al. (1983) EMBO J. 2:987-992; methotrexate, Herrera Estrella et al. (1983) Nature 303:209-213; Meijer et al. (1991) Plant Mol. Biol. 16:807-820; hygromycin, Waldron et al. (1985) Plant Mol. Biol. 5:103-108; Zhijian et al. (1995) Plant Science 108:219-227; streptomycin, Jones et al. (1987) Mol. Gen. Genet. 210:86-91; spectinomycin, Bretagne-Sagnard et al. (1996) Transgenic Res. 5:131-137; bleomycin, Hille et al. (1990) Plant Mol. Biol. 7:171-176; sulfonamide, Guerineau et al. (1990) Plant Mol. Biol. 15:127-136; bromoxynil, Stalker et al. (1988) Science 242:419-423; glyphosate, Shaw et al. (1986) Science 233:478-481; phosphinothricin, DeBlock et al. (1987) EMBO J. 6:2513-2518.

Other genes that could serve utility in the recovery of transgenic events but might not be required in the final product would include, but are not limited to, GUS (beta-glucuronidase), Jefferson (1987) Plant Mol. Biol. Rep. 5:387); GFP (green florescence protein), Chalfie et al. (1994) Science 263:802; luciferase, Teeri et al. (1989) EMBO J. 8:343; and the maize genes encoding for anthocyanin production, Ludwig et al. (1990) Science 247:449.

The transformation vector comprising the particular pathogen-inducible promoter sequence of the present invention, operably linked to a heterologous nucleotide sequence of interest in an expression cassette, can be used to transform any plant that is susceptible to downey mildew disease. In this manner, genetically modified plants, plant cells, plant tissue, seed, and the like can be obtained. Transformation protocols can vary depending on the type of plant or plant cell, i.e., monocot or dicot, targeted for transformation. Suitable methods of transforming plant cells include microinjection, Crossway et al. (1986) Biotechniques 4:320-334; electroporation, Riggs et al. (1986) Proc. Natl. Acad. Sci. USA 83:5602-5606; Agrobacterium-mediated transformation, see for example, Townsend et al. U.S. Pat. No. 5,563,055; direct gene transfer, Paszkowski et al. (1984) EMBO J. 3:2717-2722; and ballistic particle acceleration, see for example, Sanford et al. U.S. Pat. No. 4,945,050; Tomes et al. (1995) in Plant Cell, Tissue, and Organ Culture: Fundamental Methods, ed. Gamborg and Phillips (Springer-Verlag, Berlin); and McCabe et al. (1988) Biotechnology 6:923-926. Also see Weissinger et al. (1988) Annual Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Datta et al. (1990) Biotechnology 8:736-740 (rice); Klein et al. (1988) Proc. Natl. Acad. Sci. USA 85:43054309 (maize); Klein et al. (1988) Biotechnology 6:559-563 (maize); Klein et al. (1988) Plant Physiol. 91:440444 (maize); Fromm et al. (1990) Biotechnology 8:833-839; Hooydaas-Van Slogteren et al. (1984) Nature (London) 311:763-764; Bytebier et al. (1987) Proc. Natl. Acad. Sci. USA 84:5345-5349 (Liliaceae); De Wet et al. (1985) in The Experimental Manipulation of Ovule Tissues, ed. G. P. Chapman et al. (Longman, New York), pp. 197-209 (pollen); Kaeppler et al. (1990) Plant Cell Reports 9:415-418; and Kaeppler et al. (1992) Theor. Appl. Genet. 84:560-566 (whisker-mediated transformation); D. Halluin et al. (1992) Plant Cell 4:1495-1505 (electroporation); Li et al. (1993) Plant Cell Reports 12:250-255 and Christou et al. (1995) Annals of Botany 75:407413 (rice); Osjoda et al. (1996) Nature Biotechnology 14:745-750 (maize via Agrobacterium tumefaciens); all of which are herein incorporated by reference.

The cells that have been transformed can be grown into plants in accordance with conventional ways. See, for example, McCormick et al. (1986) Plant Cell Reports 5:81-84. These plants can then be grown, and pollinated with the same transformed strain or different strains.

The first step of the process of locating and identifying the pathogen inducible promoters of the invention involves isolating total RNA from a first set of plant tissue in culture which are infected with spores from P. tabacina, and from a second set of control plant tissue that are not infected. The term “infected” refers to exposing plant tissue to a finite concentration of P. tabacina spores for a predetermined length of time. Preferably, RNA is isolated from tobacco plants that have been exposed for 24 hours to 50,000 spores of P. tabacina per milliliter of MS plant medium. Total RNA is isolated by using Ambion's RNAqueous™ total RNA isolation kit, for example, and Clontech's SMART™ PCR cDNA Synthesis Kit to generate “tester” and “driver” cDNAs needed for suppression subtractive hybridization (SSH). The “tester” cDNA represents the genes that are differentially transcribed in vivo in infected plant tissue, and “driver” cDNAs represent the genes that are expressed in the control plant tissue. It is preferable to use a method for synthesizing cDNA that enriches for full-length DNAs that can be used directly in the SSH step of the cloning process described below.

Tester and driver cDNAs, are hybridized, and the hybridized sequences removed. The remaining unhybridized cDNAs represent genes that are expressed in the tester, but are absent from the driver RNA. The subtractive hybridization is performed using Clontech's PCR-Select™ cDNA Subtraction kit, and followed by suppression PCR, for example, which prevents the amplification of undesirable molecules, while enriching for target molecules (protocol number PT1117-1). The end products of the secondary PCR reaction are ligated into the pTOPOll-T/A plasmid cloning vector (Invitrogen) and used to transform E. Coli cells. Plasmid DNA is prepared from positive white bacterial colonies growing on X-gal bacterial media plates. The differentially expressed cDNAs are identified by sequence and hybridization analysis as described below.

To confirm that the individual clones represent differentially expressed genes, differential screening is performed. Duplicate dot blot arrays are created using DNA sequences of the subtracted clones. One dot blot is hybridized with labeled, e.g., radioactively labeled probes present in the tester cDNA population and the other dot blot was hybridized with labeled probes generated from the driver DNA population. Clones that are recognized by the tester but not by the driver represent genes that are differentially expressed in infected plants. The DNA sequences that are differentially expressed may then be used to hybridize Northern blots of RNAs from infected and control plants, thereby confirming the result the subtractive hybridization method. The differentially-expressed cDNAs are sequenced.

In the next step, the DNA sequences that are expressed differentially are used to generate labeled probes, which are in turn used to hybridize RNA isolated from plant tissues at predetermined time intervals after infection with P. tabacina. Total RNA is isolated prior to infection, and at 6, 24, 48, and 72 hours post-infection, but any preselected time intervals may be used. This procedure verifies that the cloned sequences are preferentially expressed at the early stages of infection, and that their expression occurs at high levels when compared to pre-infection levels. In the event that only a partial sequence of the cDNAs for the differentially-expressed gene is obtained in the cDNA synthesis step, rapid amplification of cDNA ends (RACE) may be used to clone the corresponding full-length cDNAs by extending the known sequence in either or both 5′ and 3′ directions. This method allows for cloning full-length cDNAs without screening cDNA libraries. The skilled practitioner will recognize that variations of this procedure may be used.

The next step in the process of obtaining pathogen induced promoters includes cloning the putative promoter sequence of each of the genes that were shown to be expressed differentially. This step may be performed by using Clontech's GenomeWalker™ kit and following the manufacturer's instructions (protocol number PT3042-2), although the skilled practitioner may use any suitable cloning method. First, high molecular weight tobacco genomic DNA is prepared. A plurality of aliquots of the genomic DNA are digested with one of several different restriction enzymes, and the resulting genomic DNA fragments ligated to the Genome Walker adaptor to create different genomic libraries. A gene-specific primer, for example, GSP1 is used in combination with the manufacturer's outer adapted primer, AP1, to perform the primary PCR amplification. The product of the primary amplification is diluted and used as a template for the “nested” or secondary PCR amplification.

Secondary amplification uses a gene-specific nested primer (GSP2), and the manufacturer's nested adaptor primer (AP2). The resulting DNA fragments begin with the known sequence at the 5′ end of GSP2 and extend into the unknown genomic DNA sequence. The secondary PCR products represent the putative promoter fragments. The promoter fragments are ligated into the pGEM T/A vector, which is used to transform TOP10 E. coli. Positive clones are identified by PCR, and plasmid DNA from the positive clones is prepared and digested with EcoRl. All EcoRl fragments are ligated into pBlueScript, and sequenced using M13 forward and reverse primers.

Inserts released from the plasmid by restriction enzyme digestion are amplified by PCR and ligated into the plant expression vector p308. Vector p308 comprises a DNA sequence that encodes for the E. coli reporter gene β-D-glucuronidase (GUS). The vector is used to transform E. coli; positive bacterial colonies growing on plates containing kanamycin were used to prepare plasmid DNA, which is ultimately used to electroporate strain LBA4404 of Agrobacterium tumefaciens. Using the A. tumefaciens technology, for example, plants, such as Arabidopsis and tobacco, for example, can be transformed using vacuum infiltration, and the plants can be used to test the inducibility of the cloned promoters by a variety of pathogens.

A transformed plant may be identified and isolated by selecting or screening the plant material for traits encoded by the reporter gene present on the transforming DNA. Preferably, the reporter gene is GUS; however, other reporter genes known in the art may be used. GUS activity in transformed plant tissues can be localized by observing the blue color that is formed after the hydrolysis of the uncolored substrate 5-bromo-4-chloro-3-indolyl β-D-glucuronic acid. Alternatively, GUS activity in plant extracts can be more quantitatively assayed by a fluorimetric analysis that involves the hydrolysis of the substrate 4-methylumbelliferyl β-D-glucuronide to form a fluorescent product. Both methods are standard in the art. Identification of a transferred gene may also be detected by biochemical methods including PCR, Northern analysis, RNAse protection, etc.

The promoter sequences that are identified as described above include Peronospora tabacina inducible tobacco plant promoters having the nucleotide sequence of SEQ ID NO. 1, SEQ ID NO. 2, SEQ ID NO. 3, SEQ ID NO. 4 and SEQ ID NO. 5. The isolated promoter sequences can also be used to screen for similar pathogen-inducible promoters. For example, an isolated pathogen-inducible promoter sequence of the invention may be used to isolate a DNA sequence that hybridizes under stringent conditions with the identified promoter sequence, using hybridization stringency conditions at about 65° C., followed by washing for about one hour in 2×SSC buffer at about 65° C., then washing for about 30 minutes in 0.2×SSC buffer at about 65° C. The hybridized sequences can be sequenced and/or tested for pathogen-inducibility. Preferably, the promoter sequence identified by hybridization has a nucleotide sequence that has at least about 70% and more preferably, about 90% sequence similarity to SEQ ID NO. 1, SEQ ID NO. 2, SEQ ID NO. 3, SEQ ID NO. 4 or SEQ ID NO. 5.

The following examples are offered by way of illustration of the present invention, and not by way of limitation.

### EXAMPLE 1

Cloning of Tobacco DNAs That are Differentially Expressed in Plants Infected With P. tabacina: cDNA Synthesis and Subtraction Cloning

Plant tissue derived from four to six week old KY14 plants was cultured for 24 hours in the presence or absence of 50,000 Peronospora tabacina spores/ml. Total RNA was isolated from about 250 mg of infected (I) and control (C) plant tissue using Ambion's RNAqueous total RNA isolation kit. Total RNA was used to synthesize a population of cDNA from the infected and control RNAs using Clonetech's SMART PCR cDNA synthesis kit and according to the manufacturer's protocol. The resulting cDNA was used directly for performing cDNA subtraction according to the CLONTECH PCR-Select™ cDNA subtraction kit. The products of the secondary PCR reaction that were derived from the infected and uninfected tissue were cloned into pTOPOll-T/A cloning vector (Invitrogen), according to the manufacturer's protocol. E. coli TOP10F′ were transformed with the vector constructs, and positive white bacterial colonies were selected on X-gal/carbinicillin plates. Ninety-six colonies were picked and grown in Luria Broth (LB) containing 50 mg/ml ampicillin. Plasmid DNA was prepared, and the clones were subjected to differential screening as described below.

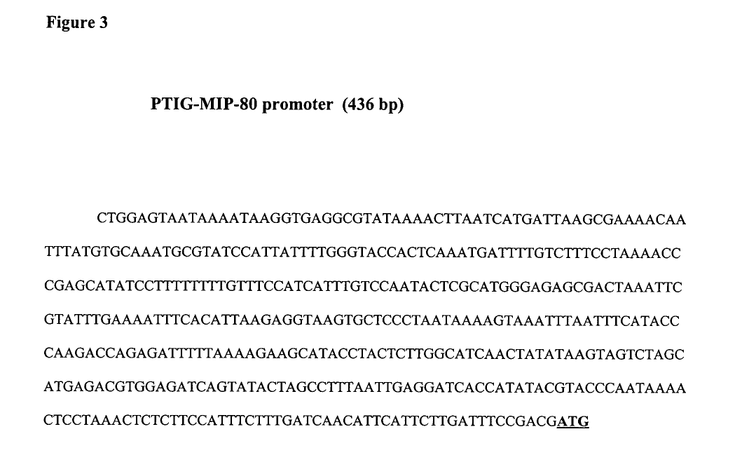

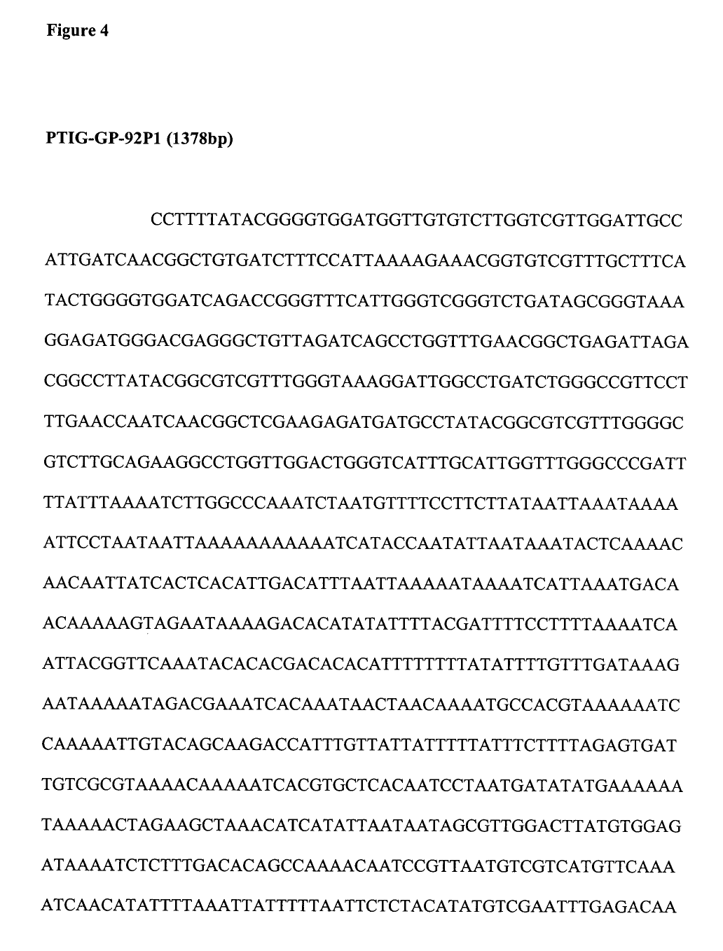

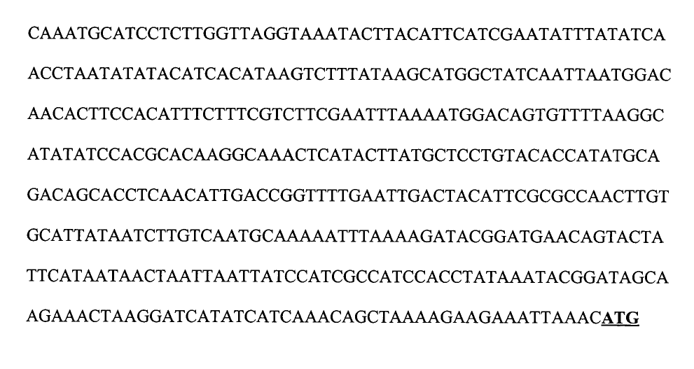

Ten microliters of each of the 96 clones was dot blotted in duplicate onto BIORAD BIODOT-Zeta Probe membranes (BIORAD), and UV crosslinked to the membranes. RNA from control and infected plant tissue was used to generate first strand cDNAs using the Superscript preamplification system of GIBCO BRL. The first strand cDNAs were radioactively labeled in the presence of ³²P-dCTP, and hybridized to the blotted clones in Church's buffer at 65° C. The membranes were washed once for 30 minutes with 2×SSC, 0.1% SDS, and three times for 30 minutes with 0.2×SSC, 1% SDS. Positive hybridization was visualized after an overnight exposure at −80° C. of the membranes to KODAK XOMAT film (FIG. 3). Twenty-four clones were identified as being specifically expressed in the infected tissue (FIG. 3), and were selected for further analysis. Clones numbered 3, 4, 11, 17, 18, 21, 22, 24, 27, 28, 30, 31, 34, 35, 37, 48, 70, 80, and 82 were digested with ECOR1 to release and visualize the cloned inserts (FIG. 4). The inserts were radioactively labeled with ³²P-dCTP and used for Northern analysis of control and infected RNA to verify the results of the differential screening. Nine of the 24 cDNAs were confirmed to be expressed differentially. Clones 11, 21, 22, 28, 30, 48, 80, 82, and 92 were sequenced using ABl/PE genetic analyzer.

Isolation of Full-Length cDNA Encoding the Isocitrate Lyase (ICL) Gene of Tobacco.

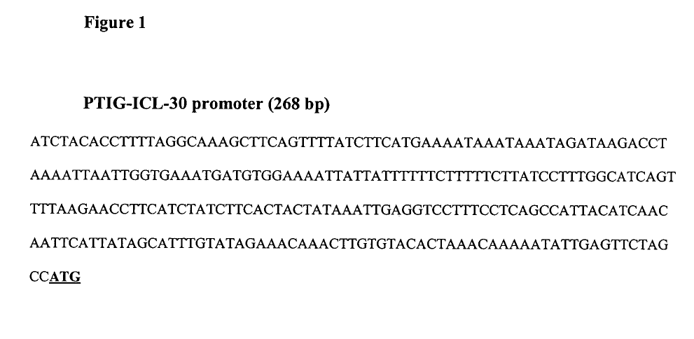

Total RNA was prepared from HAV425 plants one day after the plants had been infected for 24 hours with blue mold. First strand cDNA synthesis was performed according to Clonetech's protocol, and the RNA was reverse transcribed from an oligo dT primer using MMLV reverse transcriptase at 42° C. for 1 hour, and 94° C. for 5 minutes. PCR was performed on the first cDNA strand. Primers used to amplify the PTIG-ICL-30 promoter were: primary PCR 5′-gctcctcagtcactcctttgcccatag-3′(SEQ ID NO. 6) and secondary PCR-5′-gactgaggcaaggacctcaatttatag-3′ (SEQ ID NO. 7). The PCR protocol that was followed was: 1 minute at 96° C., 30 seconds at 94° C., 30 seconds at 50° C., 1.5 minutes at 72° C. for 29 cycles, and ending the reaction at 72° C. for 5 minutes. A PCR product of approximately 1.75 Kb was visualized on an agarose gel, and extracted using a kit from Qiagen. 5′ overhangs were generated on the 1.75 Kb cDNA by extending it at 72° C. for 15 minutes in the presence of dNTPs, PCR buffer and TAQ polymerase. A portion of the product of the extension reaction was ligated into pTOPO/TA cloning vector. Top10 E. Coli were transformed, and positive clones were isolated from white bacterial colonies selected on X-gal plates. Seven positive colonies were isolated and screened by PCR using M13 forward and reverse primers (New Engand Biolabs). All seven colonies contained the 1.75 Kb DNA insert. Qualitative agarose electrophoresis revealed the insert to be of approximately 1.8 Kb. The sequence of the 268 bp insert (PTIG-ICL-30) was determined and is shown in FIG. 1 (SEQ ID NO. 1).

This method can be applied to obtain full-length sequences of other cDNAs obtained by the methods of examples 1a and 1b. A person skilled in the art will recognize that sequences of the primers for the PCR reactions will be determined according to the partial sequence of the cDNA whose full-length sequence is to be established. However, one doesn't need the full-length DNA to isolate promoters.

### EXAMPLE 1

### Identification of Protease Inhibitor (PI) Inducible Promoter Fragments

Genomic DNA was isolated from tobacco plant tissue. Aliquots of genomic DNA were digested with one of Dral, EcoRV, Pvull, and Sspl, and ligated to the Genome Walker Adaptor to generate 4 genomic libraries.

The primers for the PCR reactions described in this method are specific for obtaining the promoter sequence of the protease inhibitor. However, a person skilled in the art will recognize that primers specific for other DNA sequences can be designed, and used according to the method described herein to clone other specific promoters.

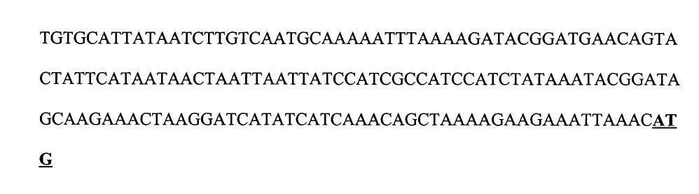

In the case of the PI gene, an antisense primer having the sequence 5′ GCC TTG GCA TCT GCA TGT TCC ACA TTG CTT AC-3′ (SEQ ID NO. 8) was synthesized based on the cDNA sequence of the protease inhibitor, and used in the primary PCR reaction. The sense primer AP1 was supplied in the manufacturer's kit. The GSP2 antisense primer having the sequence 5′-GGA CGA GAG CAA GGA AAC TAA CTC TGT GAA CAG C-3′ (SEQ ID NO. 9) was synthesized and used in combination with the manufacturer's AP2 primer to perform the secondary or nested PCR reaction. The manufacturer's protocols were followed to perform the PCR reactions. The PCR products were digested with the following restriction enzymes: Dral, EcoRV, Pvull, Scal, and Sspl, and visualized by electrophoresis on an ethydium bromide agarose gel (FIG. 6). A fragment of approximately 2.1 Kb was purified using the PCR cleanup kit from Quagen, and cloned into pGEM T/A cloning vector (Promega). Top10 E. Coli were transformed with the plasmid construct, and positive clones were selected by blue/white screening. Twenty positive clones were screened by PCR using M13 forward and reverse primers. Three of the 20 clones contained the 2.1 Kb insert. Enzyme digestion with EcoRl of the plasmid containing the 2.1 Kb insert released a first fragment of approximately 800 bp and a second fragment of approximately 1.2 Kb, which indicates the presence of an internal EcoRl restriction site. The 800 bp and the 1.2 Kb fragments were subcloned individually into EcoRl site of the vector pBlueScript. E. coli were transformed, and positive clones were selected by blue/white screening. All inserts were sequenced using M13 forward and reverse primers.

The ˜800 bp fragment of the protease inhibitor promoter was amplified by PCR using a first primer 5′Xbal PI800 having the sequence 5′-GCT CTA GAC ATT GGG GAC CTT GCT ATC GG-3′ (SEQ ID NO. 10), and a second primer 3′BahmHl PIP having the sequence 5′-GCG GAT CCC TTA CTA TCT CCT GAG TTT ACT GTT-3′ (SEQ ID NO. 11). High Fidelity Polymerase Mix (Boehringer) was used for amplification by PCR. The PCR products were purified using Qiagen PCR cleanup kit, digested with Xbal and BamHl enzymes, and ligated into the Xbal and BamHI sites of the plant expression vector p308. Twenty-nine cycles of PCR were carried out as follows: denaturation at 94° C. for 1 minute, and 94° C. for 30 seconds, annealing at 50° C. for 30 seconds, and extension at 68° C. for 1 minute. Top 10 E. Coli were transformed with the ligated constructs. Positive transformants were identified by PCR. Plasmid DNA was prepared using BIORAD's miniprep kit, and five microliters of each of the positive minipreps were used to transform Agrobacteriurn tumafaciens (LBA4404, GIBCO) by electroporation (Genepulser, BIORAD). Finally, the transformed A. tumefaciens was used to transiently transform tobacco. The expression of the GUS reporter gene was evaluated in four plants.

The 2.1 Kb promoter fragment was amplified by PCR according to the method used for the amplification of the 800 bp fragment, except that a 5′ primer 5′Xbal P12.1 having the sequence 5′-GCT CTA GAG GGA GCT CTC CCG CCT GGT CTA C-3′ (SEQ ID NO. 12) was used, and the time for extension reaction was increased to 2 minutes. The 2.1 Kb fragment was cloned into p308 as described for the 800 bp fragment, and the reporter gene was transiently expressed in tobacco as described above.

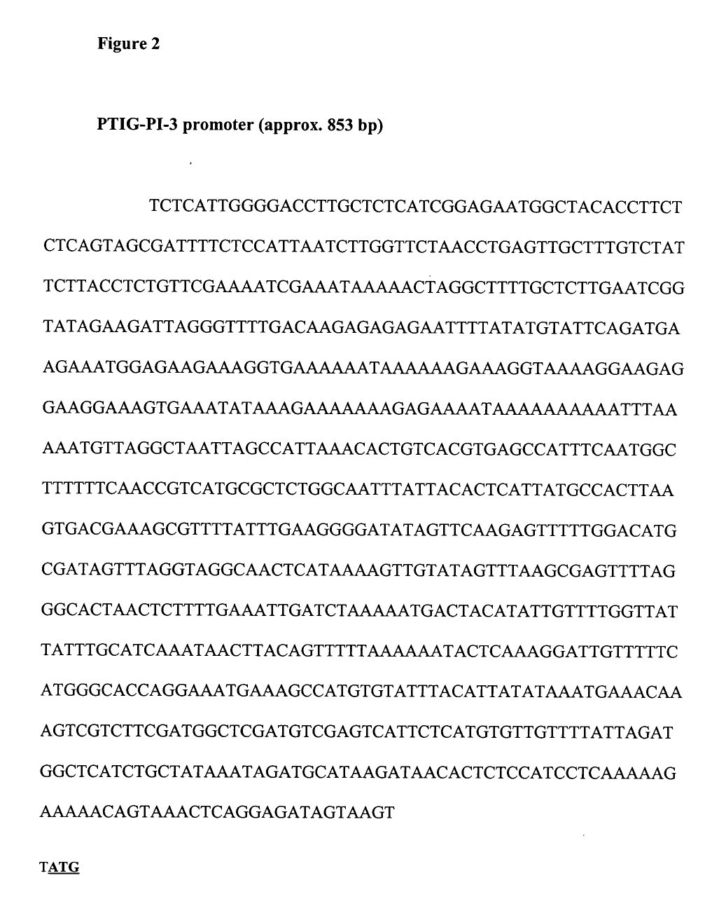

The sequence of the protease inhibitor (PI) promoter is shown in FIG. 2 (SEQ ID NO. 2).

Genome walking was also performed for identifying the promoter regions of clones 80 (SEQ ID NO. 3 and 92.(SEQ ID NO. 4 and SEQ ID NO. 5). The primers for the primary, and secondary PCR reaction of clone 80 were of the sequence: 5′-GCA AGC AAG CAA CAG AGC CAA GCA ACT GAG CA-3′ (SEQ ID NO. 13), and 5′-CAC CAA CAA ACG CAC CAAG TGA CAG CAG GGT T-3′ (SEQ ID NO. 14), respectively. The primers for the primary and secondary PCR reactions of clone 92 were of the sequence: 5′-CCC GGC TCA CAA GCT TAT TCG GGC CTG ACA A-3′ (SEQ ID NO. 15), and 5′-GGC CTG ACA AGC GGA GAG ATT GGC CCA CTA AAA GA-3′ (SEQ ID NO. 16), respectively.

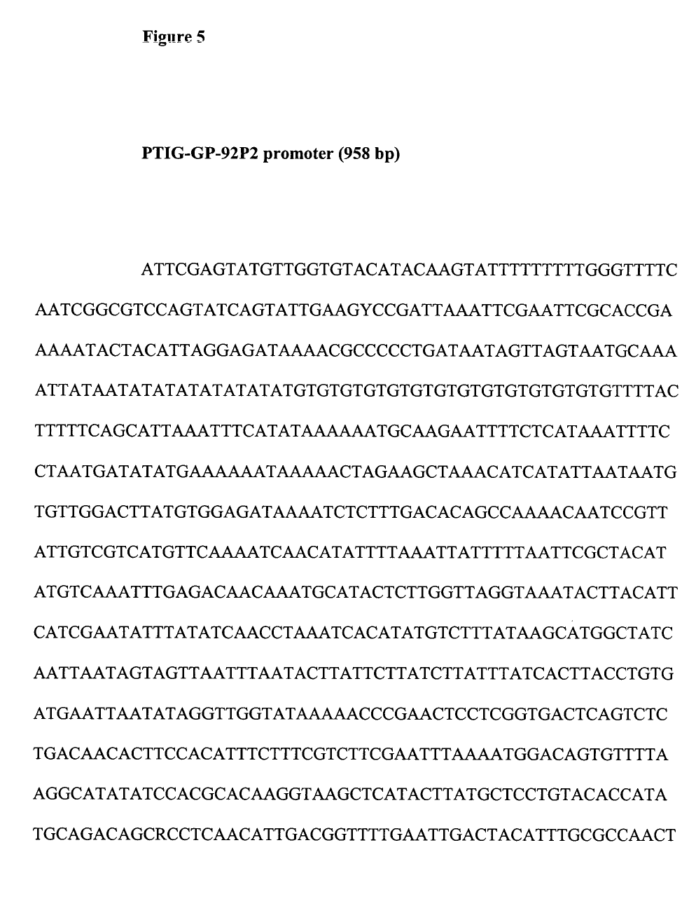

The PCR products were cloned directly into the PCR cloning vector pTOPO available from Invitrogen. The same protocol that was used for identifying the 2.1 Kb promoter fragment of PI was followed. Top 10 E. coli were transformed, and positive colonies were identified by PCR as described previously. The amplified products were visualized on agarose gels. The sequence of clone 80 is shown in FIG. 3 (SEQ ID NO. 3). Upon sequencing multiple clones derived from genome walking experiments, two different promoters were discovered corresponding to the glycoprotein gene #92 (SEQ ID NO. 4 and SEQ ID NO. 5) (FIGS. 4 and 5, respectively). These two promoters correspond at the 3′ end, but then diverge upstream.

Those skilled in the art will recognize, or be able to ascertain by routine experimentation, other embodiments according to the present invention. Other aspects, embodiments and advantages of the present invention will be apparent to the skilled person upon consultation of the foregoing description and the attached drawings and claims.

All references, including patents, patent applications and non-patent literature cited herein are expressly incorporated herein by reference.

We claim:

1. An isolated plant pathogen-inducible promoter comprising the nucleotide sequence of SEQ ID NO. 1, SEQ ID NO. 2, SEQ ID NO. 3, SEQ ID NO. 4 or SEQ ID NO. 5 or comprising a nucleotide sequence that hybridizes to the nucleotide sequence of SEQ ID NO. 1, SEQ ID NO. 2, SEQ ID NO. 3, SEQ ID NO. 4 or SEQ ID NO. 5 under high stringency conditions.

2. The promoter of claim 1 wherein the plant pathogen is Peronospora tabacina.

3. The promoter of claim 1 further comprising an enhancer sequence.

4. An expression cassette comprising a plant pathogen-inducible promoter comprising the nucleotide sequence of SEQ ID NO. 1, SEQ ID NO. 2, SEQ ID NO. 3, SEQ ID NO. 4 or SEQ ID NO. 5 or comprising a nucleotide sequence that hybridizes to the nucleotide sequence of SEQ ID NO. 1, SEQ ID NO. 2, SEQ ID NO. 3, SEQ ID NO. 4 or SEQ ID NO. 5 under high stringency conditions; a nucleotide of interest operably linked to the promoter; and a transcriptional and translational termination sequence operably linked to the nucleotide sequence of interest.

5. The expression cassette of claim 4 wherein the nucleotide sequence of interest encodes a plant defense response elicitor.

6. The expression cassette of claim 4 wherein the nucleotide sequence of interest encodes olypeptide that confers disease resistance.

7. A plant transformation vector comprising the expression cassette of claim 4.

8. A plant, plant tissue, or plant cell stably transformed with the expression cassette of claim 4.

9. The plant, plant tissue or cell of claim 8, wherein the plant, plant tissue or cell is a tobacco plant, plant tissue or cell.

10. The plant, plant tissue or cell of claim 9 wherein the expression cassette comprises a P. tabacina-inducilbe promoter.

11. A method for selectively expressing a first nucleotide sequence in a plant, the method comprising transforming the plant with a transformation vector comprising a pathogen inducible promoter and the first nucleotide sequence operably linked to the promoter, wherein the promoter is capable of initiating transcription and expression of the first nucleotide sequence in a pathogen infected plant and wherein the promoter comprises a second nucleotide sequence comprising SEQ ID NO 1, SEQ ID NO. 2, SEQ ID NO. 3, SEQ ID NO. 4 or SEQ ID NO. 5 or a nucleotide sequence that hybridizes to the nucleotide sequence of SEQ ID NO 1, SEQ ID NO. 2, SEQ ID NO. 3, SEQ ID NO. 4 or SEQ ID NO. 5 under high stringency conditions.

12. The method of claim 11 wherein the first nucleotide sequence encodes a plant defense response elicitor.

13. The method of claim 12 wherein the plant is a tobacco plant.

14. The method of claim 13 wherein the promoter is inducible by P. tabacina.

15. A method of enhancing resistance of a plant to downy mildew comprising transforming the plant with a vector comprising comprising a promoter comprising the nucleotide sequence of SEQ ID NO. 1, SEQ ID NO. 2, SEQ ID NO. 3, SEQ ID NO. 4 or SEQ ID NO. 5 or a sequence that hybridizes to SEQ ID NO. 1, SEQ ID NO. 2, SEQ ID NO. 3, SEQ ID NO. 4 or SEQ ID NO. 5 under high stringency conditions, operably linked to a gene encoding an anti-fungal agent.

16. The method of claim 10 wherein the plant is a tobacco plant.
